(19) United States
(12) Patent Application Publication
(10) Pub. No.: US 2021/0186837 A1
(43) Pub. Date: Jun. 24, 2021

(54) COSMETICALLY ACCEPTABLE ANHYDROUS COMPOSITION FOR NAIL VARNISH

(71) Applicant: INTERNATIONAL LACQUERS, Bettembourg (LU)

(72) Inventors: Frédéric Derosier, Corny sur Moselle (FR); Robin Cisneros, Saint-Julien-lès-Metz (FR); Fatima Eddoumy, Ottange (FR); Alexandre Deme, Tressange (FR); Carole Dylewicz, Terville (FR)

(73) Assignee: INTERNATIONAL LACQUERS, Bettembourg (LU)

(21) Appl. No.: 16/762,601

(22) PCT Filed: Nov. 5, 2017
(86) PCT No.: PCT/EP2018/080196

§ 371 Date: May 8, 2020

(30) Foreign Application Priority Data
Nov. 13, 2017 (FR) 1760656

(51) Int. Cl. A61K 8/49; A61Q 3/02

(52) U.S. Cl. CPC A61K 8/498; A61K 2800/31; A61Q 3/02

(57) ABSTRACT

A cosmetically acceptable anhydrous composition for nail varnish comprising at least one organic solvent, at least one film-forming agent, at least one plasticizer and at least one thixotropic agent, characterized in that it further comprises at least one nitrosamine-inhibiting thixotropic substance.

### BACKGROUND OF THE INVENTION

### Technical Field

The present invention falls within the field of the cosmetic compositions.

The invention relates in particular to an anhydrous cosmetic composition, applicable onto the nails or the false nails, which is not dangerous for the health of the users and for the environment.

### State of the Art

The nail varnishes are generally used for making up the nails as well as the false nails. The nail varnishes usually consist of a film-forming agent, a solvent, and eventually one or more plasticizers, one or more resins, one or more rheology agents, thixotropic agents, and one or more additives, namely such as dyes, pearly particles, anti-UV agents.

In order to meet the user requirements, the nail varnishes must meet several criteria:
a good application: when the nail varnish is applied onto the nail, it must form a smooth surface, without striations, with a constant thickness in order to obtain a homogeneous film and coloring,
an easy spreading over the entire surface of the nail in order to make its use practical,
an increased gloss, which persists over time,
very short drying and curing times on the nail,
a good hold of the nail varnish on the nail: it is indeed desirable that the varnish peels off as little as possible in order to maintain an aesthetic appearance.

New formulations are constantly being developed in order to try to improve any one of these properties.

In addition to the above requirements, the cosmetic products must be safe for the environment and the health of the users.

In this sense, the cosmetic industry is regulated by EC regulation No. 1223/2009 of the European Parliament and the Council of Nov. 30, 2009 relating to the cosmetic products.

Since the trend is to obtain a quality cosmetic product, meeting both the requirements of the aforementioned users and complying with said regulation, cosmetic products that are as less toxic as possible for both the environment and the users should be available.

Thus, the market for cosmetic products such as varnish must respect the wishes of the users to have quality products, without any risk to their health and the environment and containing little or no harmful or prohibited substances.

In this respect, there has been observed, within the framework of the normal use of a cosmetic product such as nail varnish, the presence, in trace amounts, of nitrosamines or nitroso-compounds.

The nitrosamines are reaction products, mostly formed unintentionally.

The nitrosamines result from the nitrosation reaction between a precursor such as a nitrosatable substrate and a nitrosating agent under acidic conditions.

“Nitrosatable substrates” means substrates such as primary amines, secondary amines, or quaternary ammonium salts, and “nitrosating agents” means substrates such as nitrites.

In the known nail varnish compositions, nitrosamine can be formed and be found in trace amounts.

Indeed, the “nitrosatable substrates” can be formed in the varnish due to its aging or also originate from its usual compounds, such as the organo-modifier present in clays.

In the same way, the “nitrosating agents” appear due to the aging of the varnish, namely due to the degradation of nitrocellulose, which is a compound frequently used to provide the varnish with gloss and a hard film.

Thus, there is a risk of formation of nitrosamines in the known nail varnishes.

According to the REACH regulation, some nitrosamines can be considered, depending on their form and their content in the product, as 1B-category carcinogens.

In particular, nitrosamines such as dimethylnitrosamines (NDMA), 1-methyl-3-nitro-1-nitrosoguanidine (MNNG), 2,2-(nitrosoimino)bisethanol (NDELA) or also nitrosodipropylamine (NDPA) are considered as toxic carcinogenic substances.

In the field of cosmetics and according to EC regulation No. 1223/2009, the nitrosamine content must not exceed 0.05 mg/kg in the various raw materials having an amine function and the maximum content of secondary amine must be less than 0.5%.

Thus, in the field of nail varnish cosmetics and according to EC regulation No. 1223/2009, it is necessary to have a safety assessment of trace amounts of prohibited substances such as nitrosamine carried out by a toxicologist in order to be able, in particular, to obtain authorizations for marketing nail varnish and for said composition to comply with EC Regulation No. 1223/2009.

As a result, in order to be able to comply with EC regulation No. 1223/2009, there is a real need to better control the presence of nitrosamines in cosmetic nail varnish compositions. In other words, during the conservation of these varnishes, it is necessary to avoid, or at least to control, the possible formation of nitrosamines over time, this without directly or indirectly modifying the intrinsic properties of the varnish. It is therefore necessary to find a solution for avoiding or limiting the formation of nitrosamines over time in a cosmetic composition of varnish without affecting all the other criteria required by the user such as gloss, coverage, hold on the nail, short drying and curing times, quick and easy spreading and application. Thus, it is necessary for the solution to be inert with respect to the usual components of the varnish and without any effect on its intrinsic properties required by the users.

### OBJECT OF THE INVENTION

Therefore, the object of the present invention is to provide an anhydrous cosmetic composition for nail varnish limiting or avoiding the formation of nitrosamines over time, while maintaining its other properties, namely good gloss, coverage, hold on the nail, as well as short drying and curing times, quick and easy spreading and application. More particularly, an anhydrous composition for nail varnish should be found, i.e. a nail varnish meeting the above criteria and properties.

### GENERAL DESCRIPTION OF THE INVENTION

The aim of the present invention is to cope with the drawbacks of the state of the art and to solve the above-mentioned problem by providing a cosmetically acceptable anhydrous composition for nail varnish comprising at least one organic solvent, at least one film-forming agent, at least one plasticizer, at least one thixotropic agent, wherein it includes in addition at least one nitrosamine-inhibiting thixotropic substance.

The term “cosmetically acceptable” should be understood as meaning a compatible cosmetic composition for application onto the human body, in particular onto the nails or the skin and which is harmless to the physical and physiological health of the user.

The term “nitrosamine-inhibiting thixotropic substance” should be understood as meaning a chemical substance, which destroys the nitrosamine already present and which also blocks its formation in the composition, and the physical flow property of which, qualified by its “thixotropic index”, varies with the time.

The thixotropic index is an indication of the rheology of the medium and its capacity to reconstitute the network permitting to guarantee the stability of the insoluble particles over time. It is generally accepted that a high “thixotropic index” guarantees greater stability over time at the level of the anti-sedimenting behavior and, at the same time, good fluidity during the application. The thixotropic index is calculated by measuring the ratio between the viscosity at 60 rpm, referred to as “V2”, and the viscosity at 6 rpm, referred to as “V3”. This V2/V3 ratio being equivalent to the use of the composition by a user, who shakes the composition of the invention before its use “V2” and who leaves it at rest “V3”.

According to the invention, the thixotropic index of the nitrosamine-inhibiting substance included in said composition is between at least 1.2 and 4.5.

Thus, said nitrosamine-inhibiting thixotropic substance permits to limit or prevent the formation of nitrosamines over time in said varnish-type cosmetic composition. As a result, any risk to the health of the user and the environment due to the presence of nitrosamine is limited. In addition, said composition will then comply with EC regulation No. 1223/2009 and be validly put on the market.

In addition, according to another feature of the invention, said nitrosamine-inhibiting thixotropic substance consists of one or more molecule(s) chosen among the molecule(s) belonging to at least one of the following families:
the hydroxypyran-4-one family, of the generic formula (I):

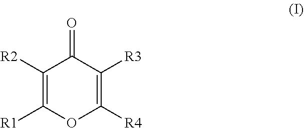

in which:
   at least one of the radicals R1, R2, R3 or R4 consists of an —OH group, or of an —OR′ group, with R′ being an ether or an ester,
   the other remaining radical or radicals consisting of an —OH, —OR′, —H group, an alkyl, an amine, an aromatic group or an aliphatic group with or without a heteroatom;
the hydroxyfuran-3-one family, of the generic formula (II):

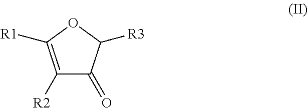

in which:
   at least one of the R1, R2 or R3 groups consists of an —OH group, or of an —OR′ group, which is an ether or an ester,
   the other remaining group or groups consisting of an —OH, —OR′, —H group, an alkyl, an amine, an aromatic group or an aliphatic group with or without a heteroatom;
the 4-hydroxymethyldioxolanone family, of the generic formula (III):

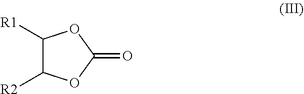

in which the radicals R1 and R2 are identical or different, R1 and/or R2 consisting of an —H group, an alkyl, an amine, an ether, an ester, an amide, an aromatic group or an aliphatic group with or without a heteroatom.

For example, the molecules of the hydroxypyran-4-one family, of the generic formula (I), can be those listed in Table 1 below:

TABLE 1

| | |
|---|---|
| 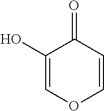 | 3-hydroxypyran-4-one |
| 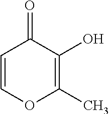 | 3-hydroxy-2-methylpyran-4-one or maltol |
| 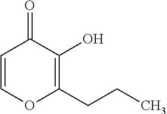 | 3-hydroxy-2-propylpyran-4-one |
| 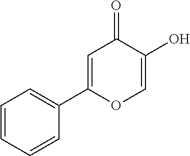 [SMILES: O=c1cc(-c2ccccc2)occ1O] | 5-hydroxy-2-phenylpyran-4-one |
| 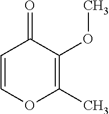 | 3-methoxy-2-methylpyran-4-one |
| 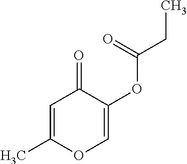 | 6-methyl-4-oxopyran-3-yl propanoate |

According to an advantageous embodiment, the molecules of the hydroxypyran-4-one family, of the generic formula (I), can have radicals R1 R2, R3 or R4 that are all identical, these radicals then being either an _OH group or an _OR′ group, as mentioned above.

According to another embodiment, the molecules of the hydroxypyran-4-one family, of the generic formula (I), can have radicals R1 R2, R3 or R4 that are all different, however at least one of them will be a radical having either an _OH group or an _OR′ group, as mentioned above.

For example, the molecules of the hydroxyfuran-3-one family, of the generic formula (II), can be those listed in Table 2 below:

TABLE 2

| | |
|---|---|
| 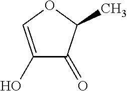 | 4-hydroxy-2-methylfuran-3-one |
| 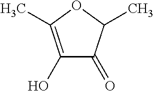 | 4-hydroxy-2,5-dimethylfuran-3-one or furaneol |
| 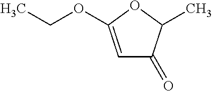 | 5-ethoxy-2-methylfuran-3-one |
| 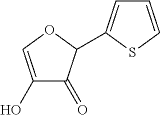 [SMILES: O=C1C(O)=COC1c1cccs1] | 4-hydroxy-2,-(thiophen-2-yl)furan-3- one |
| 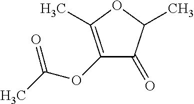 | 2,5-Dimethyl-3-oxopyran-4-yl ethanoate |

According to an advantageous embodiment, the molecules of the hydroxyfuran-3-one family, of the generic formula (II), can have radicals R1, R2 or R3 that are all identical, these radicals then being either an _OH group or an _OR′ group, as mentioned above.

According to another embodiment, the molecules of the hydroxyfuran-3-one family, of the generic formula (II), can have radicals R1, R2 or R3 that are all different, however at least one of them will be a radical having either an _OH group or an _OR′ group, as mentioned above.

According to a preferred embodiment of the invention, the molecules of the 4-hydroxymethyldioxolanone family, of the generic formula (III), can for example be chosen from the list in Table 3 below:

TABLE 3

| | |
|---|---|
| 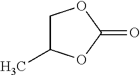 | Propylene carbonate |
| 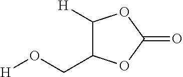 | 4-hydroxymethyl-1,3-dioxolan-2- one |
| 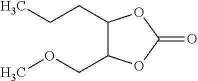 | 4-methoxymethy-5-butyl-1,3- dioxolan-2-one |
| 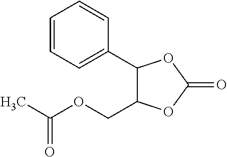 [SMILES: CC(=O)OCC1OC(=O)OC1c1ccccc1] | (2-oxo-5-phenyl-1,3-dioxolan-4- yl)methyl |

According to a preferred embodiment of the invention, said nitrosamine-inhibiting thixotropic substance consists of maltol and/or furaneol and/or propylene carbonate. Indeed, these three compounds have been specifically selected from the above-mentioned families of molecules for their specific and targeted capacities against the manufacture of nitrosamine or the elimination of same and for their inertness with respect to the other constituents of the anhydrous composition of the invention.

More specifically, each of the three aforementioned molecules taken individually has the effect of preventing the nitrosation reaction, by acting namely on the precursors such as the aforementioned nitrosatable substrates and the nitrosating agents.

In addition, each of these three molecules also permits to destroy the nitrosamine already formed within the composition. Thus, the inhibitory effect is both curative and preventive.

In addition, a synergy of the nitrosamine inhibitory effect of maltol, furaneol and propylene carbonate has been observed, which is why each of these compounds has been specifically selected as a nitrosamine inhibitor.

Also advantageously, each of these three compounds is inert with respect to the activity of the other constituents of the anhydrous composition of the invention, except for the thixotropic effect.

Indeed, advantageously, it has been demonstrated that maltol, furaneol and propylene carbonate have, in addition to their nitrosamine-inhibiting property, a thixotropic effect. The combination of these three molecules permits, within the composition of the invention, to further increase the thixotropic index, thus to further promote the stability of the composition over time.

According to a particular embodiment of the invention, said nitrosamine-inhibiting thixotropic substance represents between 0.00001% and 25% by weight in relation to the total weight of said cosmetically acceptable anhydrous composition for nail varnish. Thus, for said composition to comply with the regulation and not to be toxic to the user and the environment, only a minimal proportion of thixotropic substance, representing less than ¼ of the total composition, is necessary.

According to another feature of the invention, said anhydrous composition includes at least one organic solvent.

Said at least one organic solvent is chosen from toluene, xylene, ethyl acetate, butyl acetate, isobutyl acetate, propyl acetate, methyl acetate, a ketone, an ester, isopropanol, butanol, diacetone alcohol, ethanol denatured with methyl ethyl ketone or diethyl ether, linear or cyclic hydrocarbons, preferably cyclohexane, heptane or any combination thereof.

The organic solvent may be chosen depending on its physicochemical properties and taking into consideration the other components present in the anhydrous composition for nail varnish. Anyway, it shall be inert with respect to the thixotropic and inhibitory action of the nitrosamine-inhibiting thixotropic substance.

In addition, preferably, said at least one organic solvent represents between 10% and 90% by weight in relation to the total weight of said cosmetically acceptable anhydrous composition for nail varnish.

This amount being varying taking into consideration the other components of the anhydrous composition of the invention.

Preferably, the organic solvent is ethyl acetate, butyl acetate or a combination thereof.

According to another feature of the invention, said anhydrous composition includes at least one film-forming agent.

According to the invention, said film-forming agent is limited to the film-forming agents that can be used in cosmetically acceptable anhydrous compositions for nail varnish, i.e. to the acceptable film-forming agents known to those skilled in the art for manufacturing nail varnishes.

Said at least one film-forming agent is chosen from cellulose, a cellulose derivative, a vinyl resin, a polyester resin, an epoxy-tosylamide resin, an acrylic resin, an acrylic styrene, a copolymer resin or any combination thereof.

A film-forming agent alone or a combination of film-forming agents can be used in the anhydrous composition for nail varnish.

Advantageously, said at least one film-forming agent represents between 5 and 40% by weight in relation to the total weight of said cosmetically acceptable anhydrous composition for nail varnish.

Preferably, the film-forming agent is nitrocellulose.

According to a particular embodiment, the resins that can be used as film-forming agents are as follows:
tosylamide/formaldehyde resins or toluenesulfonamide/formaldehyde resins,
tosylamide/epoxy resins or toluenesulfonamide/epoxy resins,
copolymers of adipic acids/neopentyl glycols/trimellitic anhydrides,
copolymers of phthalic anhydrides/glycerins/glycidyl decanoates,
copolymers of phthalic anhydrides/trimellitic anhydrides/glycols,
copolymers of glycerin/phthalic acids,
polymers and copolymers of acrylates,
copolymers of acrylates and styrenes,
copolymers of styrenes/acrylates/acrylonitriles,
sucroses acetates isobutyrates,
polyvinyl butyral resins.

Preferably, the compositions contain little or no (<2%) acrylate copolymers-, acrylate and styrene copolymers- or styrene/acrylate/acrylonitrile copolymers-based compounds.

In addition to their sticky action, most of these additional resins have also a plasticizing action. The additional resins can be used in the formula, alone or in the form of mixtures, in amounts ranging from 3 to 20% by dry weight, preferably from 5 to 12% by dry weight, in particular from 7 to 10% by dry weight in relation to the total weight of the composition.

According to another feature of the invention, said anhydrous composition includes at least one plasticizer.

According to the invention, said plasticizer is limited to the plasticizers that can be used in cosmetically acceptable anhydrous compositions for nail varnish, i.e. plasticizers commonly used by a person skilled in the art for manufacturing nail varnishes.

Said at least one plasticizer is chosen from acetyl tributyl citrate, acetyl triethyl citrate, tributyl citrate, triethyl citrate, dibutyl phthalate, triphenylphosphate, triacetin, trimethyl pentanyl diisobutyrate, triethylhexanoine, sucrose benzoate, dibutyl adipate, diethyl phthalate, diisobutyl adipate, diisopropyl adipate, dipropylene glycol dibenzoate, N-ethyl toluene sulfonamide, its ortho- and para-isomers, N-(2-hydroxypropyl) benzene sulfonamide and N-(n-butyl) benzene sulfonamide or any combination thereof.

The plasticizers are compounds having a high boiling temperature, which do not evaporate when the composition of the invention such as nail varnish is drying. They are used to modulate the hardness of the film being formed, in order to obtain the desired physical-chemical characteristics of the film.

Said plasticizer represents between 1 and 20% by weight in relation to the total weight of said cosmetically acceptable anhydrous composition for nail varnish.

According to another feature of the invention, said anhydrous composition includes at least one thixotropic agent.

According to the invention, said thixotropic agent is limited to thixotropic agents that can be used in cosmetically acceptable anhydrous compositions for nail varnish, i.e. to thixotropic agents known to a person skilled in the art in the field of the manufacture of nail varnish.

Said at least one thixotropic agent chosen from pyrogenic silicas and/or hydrophilic and/or hydrophobic silicas and/or clays such as bentone, hectorite, kaolin, with or without organo-modifier(s) or any combination thereof.

Preferably, said at least one thixotropic agent represents between 0.05 and 10% by weight in relation to the total weight of said cosmetically acceptable anhydrous composition for nail varnish.

These thixotropic agents have the effect of improving the fluidity of the composition for nail varnish by maximizing its thixotropic index.

The thixotropic agent differs from the nitrosamine-inhibiting thixotropic substance in that it does not exhibit a nitrosamine-inhibiting action, but only a thixotropic action within the cosmetic anhydrous composition for nail varnish of the invention.

In contrast, the nitrosamine-inhibiting thixotropic substance has a dual action, both a “thixotropic” and a “nitrosamine-inhibiting” action.

More specifically, the nitrosamine-inhibiting thixotropic substance, namely the one belonging to the above-mentioned hydroxypyran-4-one family, of the generic formula (I), and the hydroxyfuran-3-one family, of the generic formula (II), activates the thixotropic agent(s).

In other words, the nitrosamine-inhibiting thixotropic substance, for example maltol, furaneol or propylene carbonate, and any of the possible combinations of these compounds, activates the thixotropic agent by contributing to its exfoliation, thus providing it with increased thixotropic properties.

Thus, in addition to having an effect of limiting or impeding the formation of nitrosamine over time within said nail varnish composition of the invention, said nitrosamine-inhibiting thixotropic substance acts in synergy with the thixotropic agent to promote and increase its thixotropic action. Thus, said nitrosamine-inhibiting thixotropic substance is also an activator of the thixotropic system.

According to another feature of the invention, said anhydrous composition can advantageously also include one or more additives chosen from rheological agents, UV absorbers, surface modifiers and the so-called “treating” agents and coloring agents.

For example, in order to color or provide particular effects, the anhydrous composition for nail varnish may comprise one or more coloring agents chosen from pigments, pearly particles, glitters and/or metallic particles. These coloring agents provide the aesthetic appearance looked for by the users of nail varnish. These coloring agents can produce very different effects, such as “iridescent”, “metallic”, “pearly”, “mirror”, “cream”, “glittery”, “crackled”, “ma”, “neon”, “fluorescent”, “holographic” effects, etc.

In particular, the pigments being used can be selected from the group consisting of titanium dioxide (CI 77891), black iron oxide (CI 77499), red iron oxide (CI 77491), DC Red 6 Ba Lake (CI 15850), DC Red 7 Ca Lake (CI 15850:1), DC Red 34 Ca Lake (CI 15880), FDC Yellow 5 Al Lake (CI 19140), FDC Blue 1 Al Lake (CI 42090), ultramarine blue (CI 77007), DC Violet 2 (CI 60725), DC Black 2 (CI 77266), etc.

The pearly particles can also be used in the invention. The pearly particles are mineral particles of different natures covered with one or more layers of oxides (titanium oxides or iron oxides) or also pigments. The pearly particles can be in the form of a powder or a plate of various sizes. Among the pearly particles that can be used in the invention may be cited:
the pearly particles prepared from natural mica and having been subjected to successive coating operations,
the pearly particles prepared from synthetic mica, also referred to as “synthetic fluorphlogopite”,
the pearly particles prepared from calcium sodium borosilicate,
the pearly particles prepared from silica oxide.

The glitters that can be used in the invention can be prepared from several types of material such as polyester films including a metal layer on the surface, polyethylene terephthalate, polybutylene terephthalate, acrylate copolymers, acrylate copolymers or also aluminum.

According to one embodiment of the invention, the anhydrous composition for nail varnish comprises an amount of coloring agents between 0.1% and 20% by dry weight and preferably between 0.5% and 12 to 14% by dry weight in relation to the total weight of the composition.

In addition, said composition of the invention may also include UV absorbers as additives.

Among the UV absorbers may be cited the UV light absorbents, which permit to protect the formulation or the coloring agents used in the invention against UV radiation. Among the UV protectors, which can be used in the invention, should be mentioned: benzophenone-1 (CAS 131-56-6), benzophenone-3 (CAS 131-57-7), benzyl salicylate (CAS 118-58-1), etocrylene (CAS 5232-99-5), drometrizole (CAS 2440-22-4), butyl methoxydibenzoylmethane (CAS 70356-09-1), etc.

The UV absorbers will be used alone or in the form of a combination, the total percentage of which in the formula may range from 0.1 to 1% by dry weight in relation to the total weight of the composition.

Among the additives that can be used according to the invention shall also be cited the surface modifiers such as the cyclomethicones or cyclopentasiloxanes, the dimethicones and the trimethylsiloxysilicate.

These surface additives are generally used in small amounts in the formulation, ranging from 0.1 to 5% by dry weight in relation to the total weight of the composition.

Also cited shall be the so-called additives for “treating” the nail that can be used in the composition according to the invention. The treating additives can represent from 0.01% to 5% by dry weight, preferably from 0.05% to 2% by dry weight in relation to the total weight of the composition. Among these “treating” additives shall be cited:
the formaldehyde used as a nail hardener,
the vitamin E acetate (tocopheryl acetate),
the vitamin A palmitate (retinyl palmitate),
the organic or inorganic calcium derivatives such as calcium chloride, calcium pantothenate,
dimethyloxobenzodioxasane,
myrrh,
the sulfur-containing amino acids such as cysteine, its salts and derivatives, cystine, glutathione. The sulfur-containing amino acids are indeed capable of creating crosslinking bridges between keratin and the free SH groups of its sulfur-containing derivatives,
the hydrolyzed keratin of plant origin,
the alpha-hydroxy acids such as citric acid, ascorbic acid,
biotin,
urea and dimethylurea.

The presence of all these additives permits to modulate both the aesthetic appearance of the composition and its physicochemical properties.

The present invention also relates to a nail varnish including said composition of the invention as described above.

Thus, said varnish of the invention meets EC regulation No. 1223/2009. It is safe for the consumers and the environment, it will always have a nitrosamine level lower than a value exhibiting a risk to the consumer.

In addition, the presence of the nitrosamine-inhibiting thixotropic substance has the advantage of not deteriorating the basic properties of the varnish, namely its coverage, its hold, its fluidity, its gloss, etc. On the contrary, said substance, by inhibiting nitrosamine, increases the shelf life, the storage time, the thixotropic index and the fluidity of the varnish.

The present invention also relates to a process for preparing said cosmetically acceptable anhydrous composition for nail varnish comprising at least one step in which at least one organic solvent, at least one film-forming agent, at least one plasticizer, at least one thixotropic agent are mixed with at least one nitrosamine-inhibiting thixotropic substance as mentioned above.

### Results of Tests

The results of tests shown below permit to demonstrate the effect of adding the nitrosamine-inhibiting thixotropic substance to a nail varnish composition referred to as VAO.

### Test 1:

Table 1 below shows the amount of nitrosamine present in a nail varnish composition referred to as “VAO” measured after 6 months of aging, with or without the addition of a nitrosamine-inhibiting substance.

The nitrosamines detected in the varnish are of the 2,2-(nitrosoimino)bisethanol (NDELA), dimethylnitrosamine (NDMA), N, N-diethyl-N-nitrosamine (NDEA) or also N-nitrosomorpholine (NMOR) type.

The inhibitory substances tested are furaneol, maltol or propylene carbonate and each represent 0.1% by weight in relation to the total weight of said conventional VAO composition without inhibitor.

TABLE 1

| VAO having aged for 6 months | NDELA | NDMA | NDEA | NMOR | IT (thixotropic index) |
|---|---|---|---|---|---|
| VAO without agent | 0 | 161 | 0 | 120 | 1.59 |
| VAO + 0.1% Furaneol | 0 | 145 | 0 | 92 | 1.82 |
| VAO + 0.1% Maltol | 0 | 129 | 0 | 102 | 1.89 |
| VAO + 6% propylene carbonate | 0 | 148 | 0 | 98 | 2.49 |

The results show the amount of nitrosamine measured after 6 months of aging in μg of nitrosamine/liter of varnish, as well as the thixotropic index.

A decrease in NDMA and NMOR is visible in the presence of each of the inhibitory substances being tested.

Maltol is the most effective substance to reduce the formation of NDMA, while Furaneol is the most effective substance to limit the formation of NMOR.

Furthermore, in addition to having a strong inhibiting power on NMORs, propylene carbonate has the effect of increasing the thixotropic index of VAO, hence of making it more stable.

Thus, Table 1 permits to show the thixotropic effect of inhibition of the nitrosamine of component, such as Furaneol, Maltol and propylene carbonate within a VAO.

The presence of these substances within a VAO of conventional formula has thus the effect of increasing the thixotropic index of the VAO and of reducing the toxicological risks associated with the presence of nitrosamines, this without influencing the other characteristics and properties of the varnish.

### Test 2:

Table 2 below shows the quantity of nitrosamine measured in μg/L of VAO, of a test sample, after 0, 7, 15, 18 or 31 weeks, and in which after 15 weeks was added 0.1% of Maltol by weight in relation to the total weight of the VAO test sample.

TABLE 2
Absolute values
(ppb = ug/kg = ug/L, density of VAO~1)

| Time (week) | 0 | 7 | 15 | 18 | 31 |
|---|---|---|---|---|---|
| NDELA | | | 0 | 0 | 0 |
| NDMA | 77 | 116 | 161 | 129 | 84 |
| NDEA | | | 0 | 0 | 4 |
| NMOR | 63 | 95 | 102 | 102 | 77 |

It is observed that the addition of 0.1% of Maltol permits to significantly reduce the production of NDMA and NMOR within the VAO over time.

More specifically for this test 2, Table 3 shows the percentage % of decrease in the amount of NDMA and NMOR after addition of Maltol at 15 weeks.

TABLE 3

| | % of decrease | | | | |
|---|---|---|---|---|---|
| Time (weeks) | 0 | 7 | 15 | 18 | 31 |
| NDELA | | | | | |
| NDMA | 48% | 72% | 100% | 80% | 52% |
| NDEA | | | | | |
| NMOR | 62% | 93% | 100% | 100% | 75% |

According to Table 3, Maltol has indeed an inhibitory effect on the production of nitrosamine and a destructive effect on the nitrosamine existing in the VAO.

Indeed, after 18 or 31 weeks, i.e. after addition of Maltol, the proportion of NDMA does not increase, but instead decreases, thus, Maltol prevents the formation of NDMA and destroys the NDMA that was initially present at 15 weeks.

Likewise, after 18 weeks, it is observed that the amount of NMOR is unchanged in the presence of Maltol. In other words, it is possible that Maltol will destroy as much NMOR as it produces between 15 and 18 weeks, hence a 100% stabilization. After 31 weeks, the Maltol being present destroys NMOR more quickly than NMOR is formed.

As a result, in respect to either one of the categories of nitrosamines, the addition of Maltol in an OVA has the effect of inhibiting both the nitrosation reaction and the nitrosamines.

Results similar to test 2 have been demonstrated by using propylene carbonate or furaneol instead of Maltol.

It should be noted that the effect obtained was increased in the presence of the combination of these three above-mentioned molecules.

These results prove the inhibitory and thixotropic effect of said nitricamine-inhibiting thixotropic substance present in the composition of the invention.

1. Cosmetically acceptable anhydrous composition for nail varnish, comprising:
at least one organic solvent,
at least one film-forming agent,
at least one plasticizer,
at least one thixotropic agent,
at least one nitrosamine-inhibiting thixotropic substance, which is one or more molecule(s) chosen from the molecule(s) belonging to at least one of the following families:
the hydroxypyran-4-one family, of the generic formula (I): 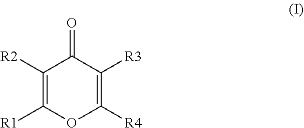
in which:
at least one selected from the group consisting of the radicals R1, R2, R3 and R4 is —OH or —OR′, R′ being an ether or an ester,
each of the other radical or radicals of the group consisting of the radicals R1, R2, R3 and R4 is selected from the group consisting of —OH, —OR′, —H, alkyls, amines, aromatic groups and aliphatic groups with or without a heteroatom;
the hydroxyfuran-3-one family, of the generic formula (II): 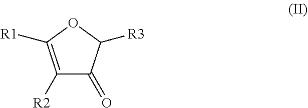
in which:
at least one selected from the group consisting of the radicals R1, R2 and R3 is —OH or —OR′, R′ being an ether or an ester,
each of the other radical or radicals of the group consisting of the radicals R1, R2, R3 and R4 is selected from the group consisting of —OH, —OR′, —H, alkyls, amines, aromatic groups and aliphatic groups with or without a heteroatom.

2. Cosmetically acceptable anhydrous composition for nail varnish according to the claim 1, wherein the nitrosamine-inhibiting thixotropic substance is one or more molecule(s) chosen from the molecule(s) belonging to the 4-hydroxymethyldioxolanone family, of the generic formula (III): 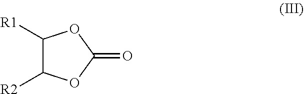
in which the radicals R1 and R2 are identical or different, and at least one selected from R1 and R2 is an —H group, an alkyl, an amine, an ether, an ester, an amide, an aromatic group or an aliphatic group with or without a heteroatom.

3. Cosmetically acceptable anhydrous composition for nail varnish according to claim 1, wherein the nitrosamine-inhibiting thixotropic substance is selected from the group consisting of maltol, furaneol, propylene carbonate, and combinations of two or more thereof.

4. Cosmetically acceptable anhydrous composition for nail varnish according to claim 1, wherein the nitrosamine-inhibiting thixotropic substance represents from 0.00001% to 25% by weight in relation to a total weight of the cosmetically acceptable anhydrous composition for nail varnish.

5. Cosmetically acceptable anhydrous composition for nail varnish according to claim 1, wherein the at least one organic solvent is selected from the group consisting of toluene, xylene, ethyl acetate, butyl acetate, isobutyl acetate, propyl acetate, methyl acetate, ketones, esters, isopropanol, butanol, diacetone alcohol, ethanol denatured with methyl ethyl ketone, ethanol denatured with diethyl ether, linear hydrocarbons, cyclic hydrocarbons, and combinations of two or more thereof.

6. Cosmetically acceptable anhydrous composition for nail varnish according to claim 1, wherein the at least one film-forming agent is selected from the group consisting of cellulose, cellulose derivatives, vinyl resins, polyester resins, epoxy-tosylamide resins, acrylic resins, acrylic styrenes, copolymer resins, and combinations of two or more thereof.

7. Cosmetically acceptable anhydrous composition for nail varnish according to claim 1, wherein the at least one plasticizer is selected from the group consisting of acetyl tributyl citrate, acetyl triethyl citrate, tributyl citrate, triethyl citrate, dibutyl phthalate, triphenylphosphate, triacetin, trimethyl pentanyl diisobutyrate, triethylhexanoin, sucrose benzoate, dibutyl adipate, diethyl phthalate, diisobutyl adipate, diisopropyl adipate, dipropylene glycol dibenzoate, N-ethyl toluene sulfonamide, ortho- and para-isomers of N-ethyl toluene sulfonamide, N-(2-hydroxypropyl) benzene sulfonamide, N-(n-butyl) benzene sulfonamide, and combinations of two or more thereof.

8. Cosmetically acceptable anhydrous composition for nail varnish according to claim 1, wherein the at least one thixotropic agent is selected from the group consisting of pyrogenic silicas, hydrophilic silicas, hydrophobic silicas, clays with organo-modifiers, clays without organo-modifier, and combinations of two or more thereof.

9. Cosmetically acceptable anhydrous composition for nail varnish according to claim 1, further comprising one or more additives selected from the group consisting of rheological agents, UV absorbers, surface modifiers so-called treating agents, coloring agents, and combinations of two or more thereof.

10. Cosmetically acceptable anhydrous composition for nail varnish according to claim 1, wherein the at least one organic solvent represents from 10% to 90% by weight in relation to a total weight of the cosmetically acceptable anhydrous composition for nail varnish.

11. Cosmetically acceptable anhydrous composition for nail varnish according to claim 1, wherein the at least one film-forming agent represents from 5 to 40% by weight in relation to a total weight of the cosmetically acceptable anhydrous composition for nail varnish.

12. Cosmetically acceptable anhydrous composition for nail varnish according to claim 1, wherein the plasticizer represents from 1 to 20% by weight in relation to a total weight of the cosmetically acceptable anhydrous composition for nail varnish.

13. Cosmetically acceptable anhydrous composition for nail varnish according to claim 1, wherein the at least one thixotropic agent represents from 0.05 to 10% by weight in relation to a total weight of said cosmetically acceptable anhydrous composition for nail varnish.

14. Nail varnish comprising an anhydrous composition according to claim 1.

15. Method for preparing a cosmetically acceptable anhydrous composition for nail varnish, comprising mixing at least one organic solvent, at least one film-forming agent, at least one plasticizer, at least one thixotropic agent with at least one nitrosamine-inhibiting thixotropic substance,
so as to obtain the cosmetically acceptable anhydrous composition for nail varnish according to claim 1.

16. Cosmetically acceptable anhydrous composition for nail varnish according to claim 5, wherein the at least one organic solvent is includes at least one hydrocarbon selected from the group consisting of cyclohexane, heptane and combinations thereof.

17. Cosmetically acceptable anhydrous composition for nail varnish according to claim 6, wherein the at least one film-forming agent is nitrocellulose.

18. Cosmetically acceptable anhydrous composition for nail varnish according to claim 8, wherein the at least one thixotropic agent includes at least one clay selected from the group consisting of bentone, hectorite, kaolin, and combinations of two or more thereof.

19. Cosmetically acceptable anhydrous composition for nail varnish according to claim 2, wherein the nitrosamine-inhibiting thixotropic substance is selected from the group consisting of maltol, furaneol, propylene carbonate, and combinations of two or more thereof.

20. Cosmetically acceptable anhydrous composition for nail varnish according to claim 2, wherein the nitrosamine-inhibiting thixotropic substance represents from 0.00001% to 25% by weight in relation to a total weight of the cosmetically acceptable anhydrous composition for nail varnish.
